(19) United States
(12) Patent Application Publication
(10) Pub. No.: US 2005/0277872 A1
(43) Pub. Date: Dec. 15, 2005

(54) Apparatus and method for mobile medical services

(76) Inventors: John Eli Colby JR., Maplewood, MN (US); Steven Craig Furlong, Maple Grove, MN (US)

Correspondence Address: ALTERA LAW GROUP, LLC, 6500 CITY WEST PARKWAY, SUITE 100, MINNEAPOLIS, MN, 55344-7704, US

(21) Appl. No.: 11/135,710

(22) Filed: May 23, 2005

Related U.S. Application Data
(60) Provisional application No. 60/574,452, filed on May 24, 2004.

(51) Int. Cl. A61N 1/18; A61N 1/32; A61N 1/38; A61N 1/40; A61N 1/26; A61N 1/10

(52) U.S. Cl. 604/67; 607/6; 607/62

(57) ABSTRACT

A mobile personal computing and communications device provides for secure, reliable, accurate and up-to-date point-of-care medical information for rendering sound medical care to a patient. The mobile personal device may be used by either the patient and/or by caregivers to furnish, access and/or acquire and store the patient's medical records using wireless communications, to provide a treatment, to facilitate compliance with a treatment plan, to facilitate patient care, to facilitate collaboration on treatment for the patient's condition, and to proffer an authenticated medical directive, among other mobile medical services.

### CROSS-REFERENCE TO RELATED APPLICATIONS

This patent document claims the benefit of U.S. Provisional Patent Application Ser. No. 60/574,452 filed May 24, 2004 (Colby et al., Apparatus and Method for Mobile Medical Services, Attorney Docket No. 01726.0008-US-P1), which hereby is incorporated herein in its entirety by reference thereto.

### BACKGROUND OF THE INVENTION

1. Field of the Invention

The present invention is directed to apparatus and method for mobile medical services, and more particularly to methods and apparatus to enhance and facilitate medical practices and procedures in a wireless and mobile environment.

2. Description of the Related Art

Mobile phones and laptop/portable computers have each separately and in conjunction increased the portability of the traditional “fixed-in-place” corporate office workspace. In many instances, this portability has increased the efficiency and accessibility of staffers by both their colleagues and clients. All corporations and organizations are looking for ways to increase productivity and ultimately profitability in their business model, and medical facilities are no exception. Physicians, nurses, emergency room staff, and a myriad of other healthcare professionals all work in an environment where timely and accurate medical information is of the utmost importance.

Given that major modern medical facilities now take on the look of a college campus spread out over many buildings and over a large footprint, the rapid and accurate transmittal of patient information can be challenging. Also, in some instances, physicians and nurses could benefit from immediate access to colleagues with a specialized expertise or access to a medical database with information relevant to the patient at hand. These types of information may be extremely useful if the physician can access the data real-time at the point of care, for example in emergency rooms or en-route in ambulances or medivacs.

### BRIEF SUMMARY OF THE INVENTION

Secure, reliable, accurate and up-to-date point-of-care medical information is essential for rendering sound medical care to a patient. In a highly controlled setting such as a hospital, for example, health care professionals are readily available to administer to the patient based on the patient's historical and present medical data. The patient's historical medical data is available from various disparate sources such as the hospital's computers, paper files, and the patient's chart, while near real time physiological data is available from a bedside monitor as well as special purpose monitors using a myriad of invasive and non-invasive sensors. The attending physician may collaborate with other physicians by telephone or from a computer terminal using the hospital's high bandwidth network. Appropriate treatments are determined by the attending physician and administered manually by a nurse or automatically by bedside equipment. Unfortunately, this life-saving technology is expensive and is impractical for transport and use outside of such highly controlled settings as a hospital. In particular, this life-saving technology is impractical for use where the point-of-care is in the home, a nursing home, assisted living facility, or other similar type of care facility, at an accident scene, or in an emergency transport vehicle such as an ambulance or aircraft.

Some aspects of this problem are solved by an embodiment of the present invention, which is a method of providing care to a patient who is provided with a personal computing device having a size, weight and form factor suitable for mobile use by the patient. The method comprises registering a patient-associated physiological monitoring device with the personal computing device during an authenticated session; securely acquiring patient physiological data from the patient-associated physiological monitoring device with the personal computing device; analyzing the patient physiological data with the personal computing device to generate a patient care recommendation; and controlling the application of a treatment in accordance with the patient care recommendation. The personal computing device may also have a wireless communications capability by which a patient care recommendation may be transmitted to a physician for approval, and for receiving the physician's approval.

Another embodiment of the present invention that solves this problem is a mobile personal computing device for providing a treatment to a patient having a patient-associated physiological monitoring device, comprising means for initiating a patient-authenticated session; means for registering the patient-associated physiological monitoring device with the personal computing device during the patient-authenticated session; means for securely acquiring patient physiological data from the patient-associated physiological monitoring device with the personal computing device; means for analyzing the patient physiological data with the personal computing device to generate a patient care recommendation; and means for controlling the application of a treatment in accordance with the patient care recommendation. The mobile personal computing device is of a size, weight and form factor suitable for mobile use by the patient.

Other aspects of the problem are solved by another embodiment of the present invention, which is a method of distributed caregiving to a patient at a point-of-care. The patient is provided with a personal computing and communications device having a wireless communications capability, and further having a size, weight and form factor suitable for mobile use by the patient. The method comprises storing first patient medical data in the personal computing and communications device at the point-of-care; acquiring second patient medical data regarding a current condition of the patient with the personal computing device at the point-of-care; acquiring third patient medical data from at least one remote caregiver in an authenticated session using the communications capability of the personal computing device, at the point-of-care; furnishing selected items from at least one of the first, second and third patient medical data to at least one remote caregiver in an authenticated session using the communications capability of the personal computing device; generating patient treatment instructions for the current condition with the personal computing device from the third patient medical data; and expressing the patient treatment instructions with the personal computing device at the point-of care.

Another embodiment of the present invention that solves this problem is a mobile personal computing and communications device for distributed caregiving to a patient at a point-of-care, comprising means for storing first patient medical data in the personal computing and communications device at the point-of-care; means for acquiring second patient medical data regarding a current condition of the patient with the personal computing and communications device at the point-of-care; wireless communications means for acquiring third patient medical data from at least one remote caregiver in an authenticated session at the point-of-care, and for furnishing selected items from at least one of the first, second and third patient medical data to at least one remote caregiver in an authenticated session; means for generating patient treatment instructions for the current condition with the personal computing and communications device from the third patient medical data; and means for expressing the patient treatment instructions with the personal computing and communications device at the point-of care. The mobile personal computing and communications device is of a size, weight and form factor suitable for mobile use by the patient.

The problem of requesting assistance in the event of a possible traumatic event is solved in another embodiment of the present invention, a mobile personal computing and communications device comprising a unit having a size, weight and form factor suitable for mobile use by the user; a processor capability contained within the unit and having sufficient processing power for executing computer programs that are designed for personal computers; a user interface functionally integrated with the processor capability; a cellular communications capability contained within the unit; a broadband wireless communications capability contained within the unit; a wireless personal area network capability contained within the unit; a security capability contained within the unit and functionally integrated with the processor capability, the cellular communications capability, and the broadband wireless capability for providing an authenticated login at the user interface and secure wireless communications with at least the broadband wireless communications capability; a global positioning system capability functionally integrated with the processor capability; an optical scanner capability functionally integrated with the processor capability; a camera capability functionally integrated with the processor capability and the cellular communications capability; a multiple-axes accelerometer functionally integrated with the processor capability and tightly mechanically coupled to the unit; and a storage medium readable by the processor subsystem and comprising executable code. In a variation, the storage medium may comprise executable code for detecting via the accelerometer acceleration of the unit greater than a first threshold indicative of possible injury to a user of the personal computing and communications device; detecting failure of the user to indicate no injury via the user interface in response to the first threshold acceleration detection step; and requesting assistance in response to the failure detecting step,- using any of the cellular communications capability, the broadband wireless communications capability, and the wireless personal area network capability.

The problem of authenticating a medical directive of a person that is proffered after the person is incapacitated is solved in a method that comprises initiating a first authenticated session on a personal computing device with a biometric of the person, the personal computing device having a size, weight and form factor suitable for mobile use by the person; storing a medical directive on a personal computing device during the patient-authenticated session; during the patient-authenticated session, securing the stored medical directive for access by a proxy, the proxy being identified by a biometric of the proxy; initiating a second authenticated session on the personal computing device with the biometric of the proxy; and expressing the secured medical directive during the second authenticated session. In a variation, the credibility of the medical directive may be adjudicated from the secured medical directive and a confirmation version thereof on remote storage.

Another embodiment of the present invention that solves this problem is a personal computing device for authenticating a medical directive of a person that is proffered after the person is incapacitated, comprising means for initiating a first authenticated session on the personal computing device with a biometric of the person; means for storing a medical directive on a personal computing device during the patient-authenticated session; means for securing the stored medical directive during the patient-authenticated session for access by a proxy, the proxy being identified by a biometric of the proxy; means for initiating a second authenticated session on the personal computing device with the biometric of the proxy; and means for expressing the secured medical directive during the second authenticated session. The personal computing device has a size, weight and form factor suitable for mobile use by the person.

### BRIEF DESCRIPTION OF THE SEVERAL VIEWS OF THE DRAWINGS

The invention may be more completely understood in consideration of the following detailed description of various embodiments of the invention in connection with the accompanying drawings, which are as follows.

FIG. 1 is a block diagram of an embodiment of a mobile personal computing and communications device for medical applications, in accordance with the present invention.

FIG. 2 is a perspective view showing various aspects of an embodiment of a mobile personal communication device for medical applications, in accordance with the present invention.

FIG. 3 is a perspective view showing various aspects of an embodiment of a mobile personal computing and communications device for medical applications, set into a docking cradle, in accordance with the present invention.

FIG. 4 is a sketch showing a variation of the mobile personal computing and communications device of FIG. 3, in accordance with the present invention.

FIG. 5 is a sketch showing another variation of the mobile personal computing and communications device of FIG. 3, in accordance with the present invention.

FIG. 6 is a flowchart showing a method useful for a medical worker to prepare for a patient visit while the medical worker in route to the patient, in accordance with the present invention.

FIG. 7 is a flowchart showing a method useful for facilitating compliance with a patient treatment plan, in accordance with the present invention.

FIG. 8 is a flowchart showing a method useful for facilitating care of a patient, in accordance with the present invention.

FIG. 9 is a flowchart showing a method useful for providing a treatment to a patient, in accordance with the present invention.

FIG. 10 is a flowchart showing a method useful for distributed caregiving to a patient, in accordance with the present invention.

FIG. 11 is a flowchart showing a method useful for proffering an authenticated medical derivative of a person, in accordance with the present invention.

FIG. 12 is a sketch showing a variation of the mobile personal computing and communications device of FIG. 4, in accordance with the present invention.

### DETAILED DESCRIPTION OF THE INVENTION, INCLUDING THE BEST MODE

While the invention is amenable to various modifications and alternative forms, specifics thereof are shown by way of example in the drawings and described in detail herein. It should be understood, however, that the intention is not to limit the invention to the particular embodiments described. On the contrary, the intention is to cover all modifications, equivalents, and alternatives falling within the spirit and scope of the invention.

Methods and apparatus are described herein that enhance and facilitate medical practices and procedures in a wireless and mobile environment, especially where an integrated mobile phone and computer is utilized with a secure network services, whether or not dedicated, to perform these functions. While a variety of mobile personal devices and networks may eventually be developed with suitable capabilities, one such device and network, along with related connections and communications management, are described in copending U.S. patent application Ser. No. 10/849,269, filed May 19, 2004, which names inventor John Eli Colby, Jr. and is entitled “Apparatus and method for mobile personal computing and communications” and which hereby is incorporated herein in its entirety by reference thereto.

Surgical teams, and their support groups, represent one area within the hospital where the present invention may be extremely useful. For example, a cardiac emergency response team such as a cardiac by-pass surgery team could be quickly called into action to respond to a potentially life threatening situation such as an emergency stat condition or a “code blue” situation if each member of the team carried with them a portable integrated mobile phone and computer with a secure network service to provide on-demand relevant patient information. To be accessible on-demand, the patient information may be available from local storage on the mobile personal device, or may be retrieved from a remote server or storage medium in real time or with minor delay because of temporary network outages due to traffic or switching between networks. Seamless networking and session persistence makes the process of acquiring patient information from remote sources transparent to the user, even if networks are changed during acquisition of the patient information.

In the above scenario, the cardiac emergency response team is able to access through the wireless network services, relevant medical history information even while en-route to the hospital. This information may include, but is not limited to: patient's prior history of cardiac events and outcomes, patient's blood type; lab results; current medications patient may be taking; patient's known allergic reactions; and next of kin to authorize treatment (e.g., if patient is unconscious). In addition to access of this archived patient data, real-time or near real-time point-of-care data may be paramount in this type of emergency situation. For example, paramedics arriving on the scene of an unconscious patient may acquire vital signs information (blood pressure, heart rate, blood oxygen saturation, temperature, and possibly EEG data and so forth) and provide this data to the converging emergency response team via the medical information network. Similarly, data taken in the ambulance in route to the hospital can be shared in near real-time with both the converging emergency response team and the admitting staff at the hospital emergency room to prepare for patient arrival. Inasmuch as the sharing of information between separated persons necessarily involves some delay, the term “near real-time” includes extremely small delays imperceptible to the recipient as well as longer delays perceptible to the recipient, provided the delayed information is still relevant to the patient's current condition.

Also, if the mobile computer and phone system has wireless video receive and transmit capabilities, the en-route medical professional can receive and review X-rays, MRI's, PET Scans, and other relevant imaging data and may video teleconference with other colleagues for a confirming or second opinion prior to choosing a course of action for the patient.

Of course, the above scenario may also be implemented within the hospital or medical facility where key “code blue” or emergency medical response team staff are distributed within multiple departments, for example, emergency room staff, surgery team, catheter lab, blood lab personnel, paramedics en route, and so forth, all of which could be immediately called into action and have their efforts coordinated through networked mobile personal devices.

While personal mobile devices may be carried by medical staff for communication amongst themselves while responding to an emergency event, a personal mobile device may be carried by an ordinary person, who has log-on access to communicate with a primary care physician (or cardiologist for this example) through any suitable network. For example, a patient who may be experiencing severe chest pains may initiate a “911” type request for immediate medical assistance utilizing their personal mobile device. The patient's personal mobile device may be configured to automatically transmit its “GPS” coordinates so that the emergency response medical team or other responding emergency service knows the patient's location. In addition, the patient's personal mobile device may transmit other information about the patient, ranging from historical patient medical data to current physiological data, so that the emergency medical response team, whether en route to the patient or awaiting the patient's arrival at a hospital, may prepare better to deal with the medical emergency. In addition, the patient's personal mobile device may transmit other information such as images of the accident scene to better prepare the emergency responders. In this scenario, the patient may be in either constant, or periodic, or event-driven communication with medical personnel, such as the emergency medical response team members, primary care physician, cardiologist, emergency room physicians, and so forth, while en-route to the hospital.

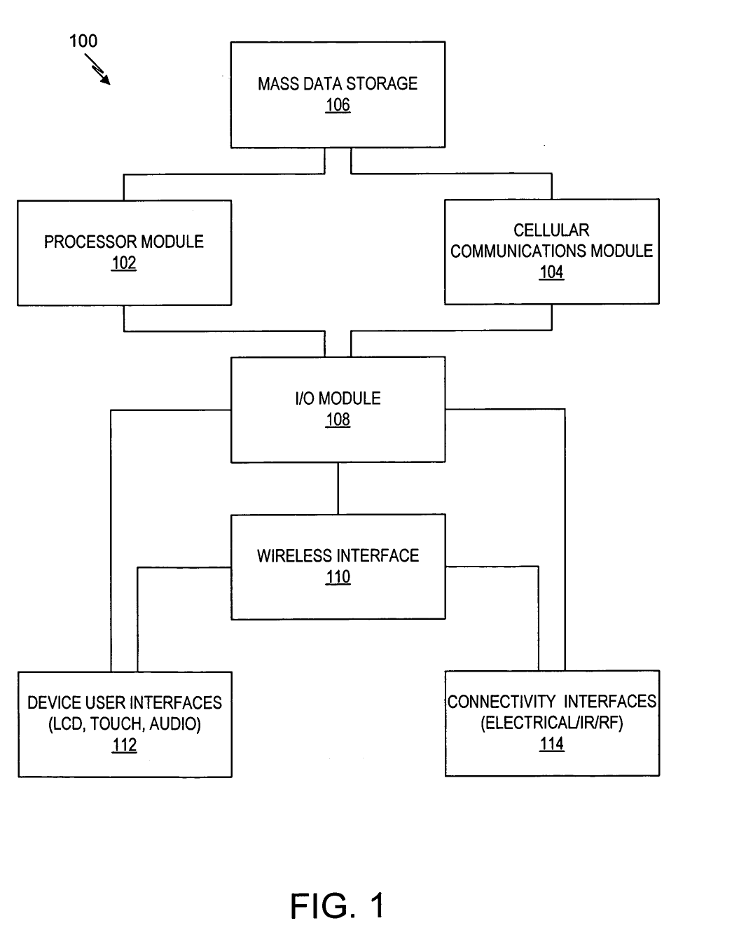

The generic features of an illustrative mobile personal device 100 and associated network services suitable for implementing the above scenario are described below, with reference to the embodiments of FIG. 1 through FIG. 5. However, the general attributes of a suitable mobile personal device are as follows.

A suitable mobile personal device preferably is a combination of a cell phone and a full function personal computer, along with sophisticated connection and communications management to form an integrated, mobile, wireless, handheld end-user computing and communication device. A suitable mobile personal device may also have the ability to capture, store, and transmit video images as well as accept verbal commands utilizing speech recognition software. A suitable mobile personal device may also have an integrated bar code reader and/or optical scanner with character recognition capability. A suitable mobile personal device is capable of providing essentially the equivalent connectivity and computing capabilities of a full desktop/laptop computer with wireless Local Area Network/Wide Area Network (LAN/WAN) and cellular connection. A suitable mobile personal device may also have “Bluetooth” wireless technology or other short range connection technology incorporated therein for implementing a Personal Area Network (PAN) for close proximity wireless attachment of accessories such as headset, mouse, keyboard, printer, implantable medical devices, subcutaneous and transcutaneous medical devices, non-invasive medical devices, and so forth. “Bluetooth” technology and proprietary protocols may also be used for short-distance wireless LAN applications. Wi-Fi (Wireless Fidelity) and cellular technologies may provide the system network connection for all other LAN and WAN applications

For the medical application environment, secure Wi-Fi is preferred as the primary wireless network connection and data transmission methodology. Cellular networks may be utilized beyond the office/enterprise environment when no broadband connection is available to provide the seamless mobile voice and data network connectivity.

For the medical professional, a suitable mobile personal device provides a single device for mobile computing and communications that is wirelessly connected to the Medical Information Network for seamless connectivity and timely updates from all other associated information systems inside and outside the network. These information systems may include, but are not limited to, laboratory results, insurance information, patient records, radiology information, personnel information, physician/professional services, pharmacy, patient scheduling, messaging (email and calendar), Internet/World Wide Web access, Internet Service Provider (ISP) connectivity, subscription-based data services, accounts receivable, customer service, and related systems from other clinics or hospitals. A suitable mobile personal device provides logon security, network security, information and data security, and network access control through secure Wi-Fi connection, data encryption, and cellular identification technologies and methods.

A suitable mobile personal device is a small compact portable device that can be carried like a cell phone or PDA. A small display and a thumb-operated keyboard or a virtual stylus operated keyboard are incorporated into the device for mobile user convenience. However, when the medical professional is working on the desktop or in other normal workstation environments, a suitable mobile personal device may be placed into a docking station thereby connecting to a larger display, full size keyboard, desktop mouse, and other devices or network connections. For the mobile medical professional, a suitable mobile personal device may become the true personal communications and computing device that is “always connected everywhere” to information systems, the internet, and cellular networks for information, data processing, and voice communications. Currently each individual device must have its own Personal Information Manager (PIM) which requires frequent synchronization or system replication and creates the need for a complex support structure by the facility's Information Systems (IS) staff. Because a suitable mobile personal device is fully integrated, user-specific information stored within the device is managed by the internal software and is shared among the computer and cell phone functions.

FIG. 1 is a schematic block diagram of one example of a suitable architecture of a suitable mobile personal device 100, which is described in the aforementioned copending Colby patent application. The device 100 is arranged as internal modules, although this arrangement is illustrative and other device architectures may be used. In particular, advanced fabrication technologies permit the various discrete functions to be implemented on one or more highly integrated substrates. The processor module 102, which carries out the major processing functions, includes a microprocessor chip set and associated memory, and is capable of running a standard type of operating system that normally runs on a desktop or laptop computer, such as Windows XP or equivalent. Accordingly, the processor module 102 may run many different types of software applications, just like laptop and desktop computers. This computing capability is different from that of a cell phone that has a PDA capability, or a miniaturized computer that runs a reduced operating system such as Windows CE. Such computers are not capable of operating the same software applications as are normally run on laptop or desktop computers, such as full versions of the Microsoft Office suite of business software tools that includes word processing, spreadsheet, and presentation applications.

A cellular communications module 104 provides cellular communications capabilities, and allows the device 100 to operate as a cell-phone. A mass data storage unit 106 provides data storage for at least the processor module 102. The mass data storage unit 106 may be any suitable type of mass data storage unit, for example a magnetic storage unit, and preferably provides read/write data storage capabilities. One example of a mass data storage unit is a magnetic hard disk, although other types of data storage may also be used. The mass data storage unit preferably provides permanent data storage, that is storage of data even when power is removed. Permanent data storage does not require that the data be unerasable.

The mass data storage unit 106 contains enough capacity to enable the mobile personal to run business software applications normally run on a laptop or desktop computer, and preferably has a capacity of 2 Gigabytes or greater. This compares with currently available hand-held devices where the storage capacity is approximately 32 MB. This additional storage capacity enables users to replicate the same functionality as current laptop or desktop computers without the associated weight or bulk of those larger devices. This additional capacity enables new applications, for example “lifetime phone conversation recording”, in which all telephone conversations taking place on the device 100 may be recorded over its lifespan. Furthermore, because the mass data storage unit saves data permanently, the information is not lost if battery power to the mobile personal device is lost.

The processor module 102 and cellular communications module 104 communicate via an I/O module 108 to various I/O devices having either wired or wireless connectivity. For example, a wireless interface module 110 is provided to allow for wireless communication.

Various device user interfaces 112 may be provided on the device 100 itself; for example, an LCD display to display information to the user, a touch screen overlying the LCD display as an input device to receive information form the user, and audio input and output channels. The audio input channel may include a microphone to receive audio input from the user, and the output channel may include a speaker to furnish sound to the user. The audio channels are useful for phone conversations over the cellular communications module 104, including cell phone speakerphone communications. The audio channels may also be used to furnish commands for voice activated processes and data for processes run by the processor module 102 (for example, voice recognition for generating alphanumeric commands and data from oral communications), and to communicate information in audio form (for example, using a text to audio translator) from the running operating system and applications.

The device 100 may also have one or more interfaces 114 for connection to peripheral devices, such as a docking station, external keyboard, external monitor, and the like. The interfaces 114 may include, for example, various sockets for plugging peripherals into the device 100. An example of a suitable interface is the universal serial bus, or USB interface. Other examples include a microphone jack, a headphone jack for private audio output, and a multiconnector jack for a hands free headset. The interfaces 114 may also include wireless connectivity to establish a personal area network, including connectivity options such as Bluetooth, infrared, and so forth. The interfaces 114 may also include wireless connectivity to local area networks, including Wi-Fi connectivity. Suitable Wi-Fi technology includes 802.11(a)(b)(g), which provides Ethernet network connectivity incorporating any secure wireless authentication protocol such as LEAP. The interfaces 114 may include proprietary connectivity options as well, such as connectivity to proprietary sensors to sense signals from proprietary devices such as implanted as well as noninvasive medical devices (including subcutaneous and transcutaneous), security tags, and so forth.

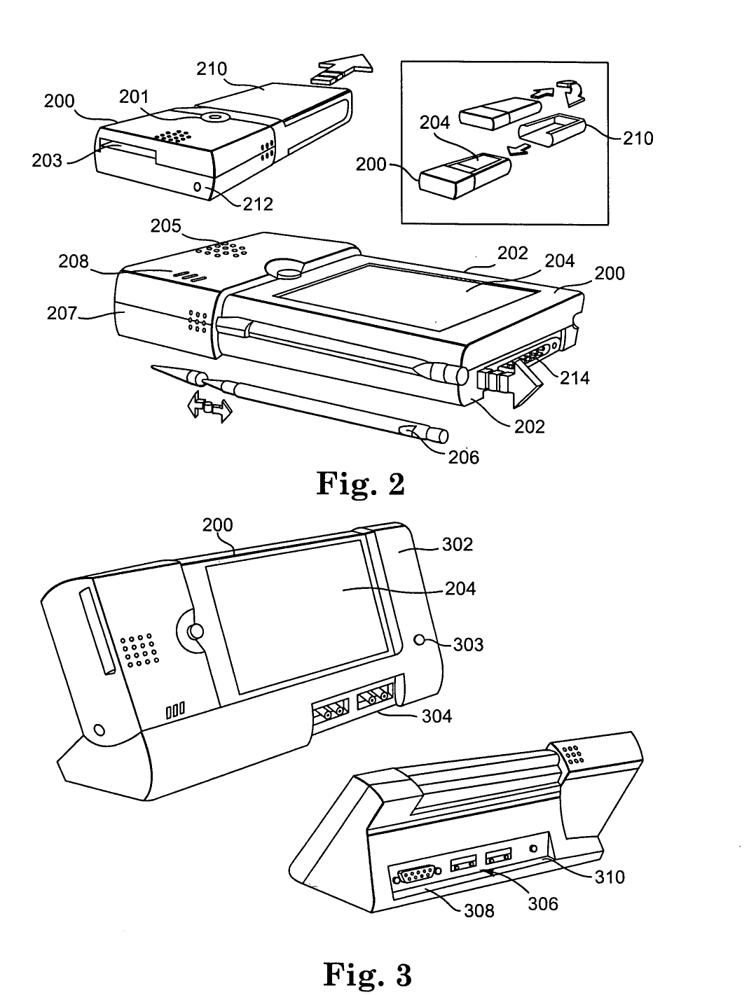

An illustrative embodiment of a mobile personal device 200 is illustrated in several perspective views in FIG. 2. The device 200 has on/off switch 201, and is housed within a casing 202 having a rubber overmold section 207 with molded texture 205 for secure handling. The device 200 has a screen 204 on one side, that may be a touch sensitive screen for displaying information to the user and for receiving information input from the user. A stylus 206, provided with the device 200, may be used for inputting information to the device 200 via the touch screen 204. The stylus 206 may be convertible into a pen for the user to use for writing. The device 200 may be provided with a built in microphone 208, with access holes through the casing 202 to receive audio signals at the microphone. The device may also include a speaker (not shown) for sending audio signals to the user. A media slot 203 may be provided for insertable/removable memory media such as Secure Digital (“SD”), SmartMedia, and Memory Stick.

The device 200 may also be provided with a protective cover 210 that may be placed over the screen 204 to provide protection when the device 200 is not in use, and that can be removed to reveal the screen 204 when the user desired to use the screen 204. In one embodiment, the cover 210 may be reversible, so that when it is not covering the screen 204, the cover 210 covers the backside of the device 200, as is illustrated in the inset in FIG. 2.

The device 200 may also have an electrical input 212 to receive electrical power from, for example, an AC adapter for operating, recharging, or both. The device 200 is also provided with a capability for external connections, such as, for example, peripheral devices, a network, and so forth. In the illustrated embodiment, such connectivity includes electrical connections 214 that may be used for wired connections such as, for example, a network connector for connection to a network, a video connector for connection to an external display screen, a keyboard connection for connection to an external keyboard, and so forth.

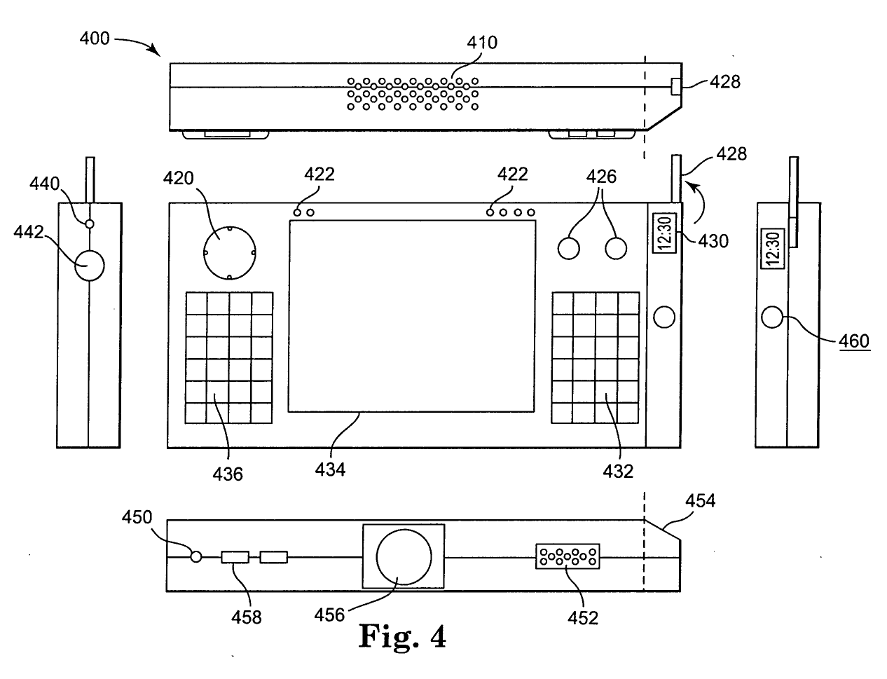

Alternatively, the connections 214 may be designed for connecting the device 200 to a customized docking station 302, an example of which is illustrated in several perspective views in FIG. 3. The docking station 302 may provide additional connectivity to input devices such as, for example, a keyboard, mouse, joystick and so forth via inputs 304, and to output devices such as monitors, printers and so forth, via outputs 306. The input and outputs 304 and 306 may be USB ports or any other suitable type of port. For example, a video port 308 may be used to connect to an external video monitor. The docking station 302 may also provide connectivity to a network via, for example, an Ethernet connection.

The docking station 302 may also be used for recharging the battery or batteries in the device 200. Consequently, the docking station 302 is provided with input 310 to receive electrical power from, for example, an AC adapter. LED 303 indicates the power status of the device 200. As is illustrated, the docking station may advantageously orient the device 200 so that the user is able to view the screen 204, although this is not a necessary feature of the docking station 302.

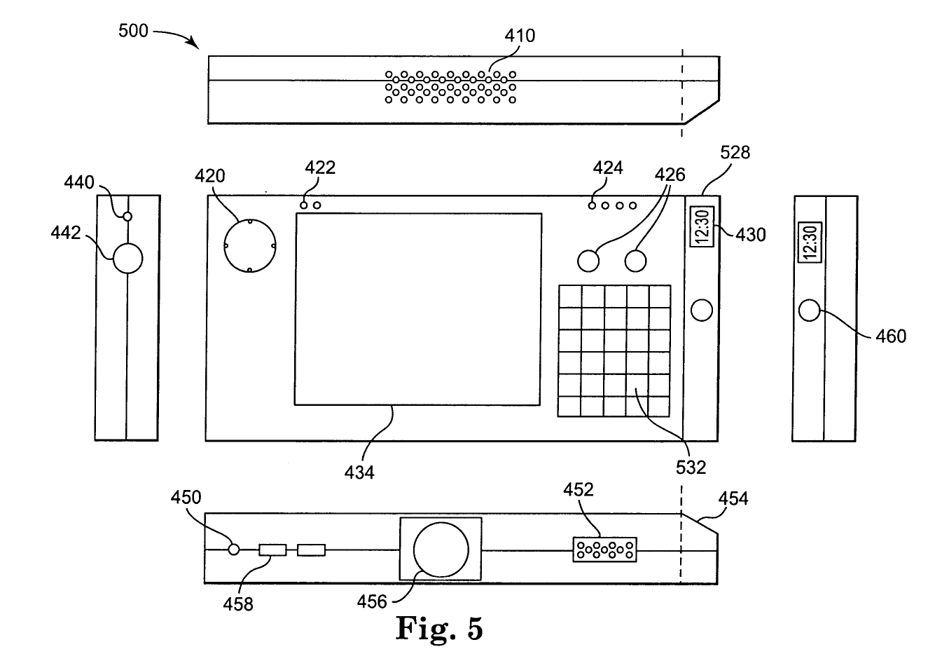

FIG. 4 is an illustration of an alternative platform configuration of a mobile personal device 400 that has a split onboard keyboard. The device 400 has cooling vents 410 and a foldable GSM antenna 428 along a top edge, a headset jack 440 and a power button 442 along a left edge, and a phone power button 460 and a one inch LCD phone screen 430 along a beveled portion 454 of the right edge. The bottom edge of the device 400 includes a charging jack 450, a docking connector 452, a cooling fan 456, and two USB connectors 458. A front face of the device 400 includes a mouse navigation pad 420, LED indicators 422 for power and other device status information, speaker and microphone vents 424, left and right buttons 426 configurable for use with the mouse pad 420, and a five inch LCD main display screen 434. The front face of the device 400 also includes a split keyboard having right and left sections 436 and 432.

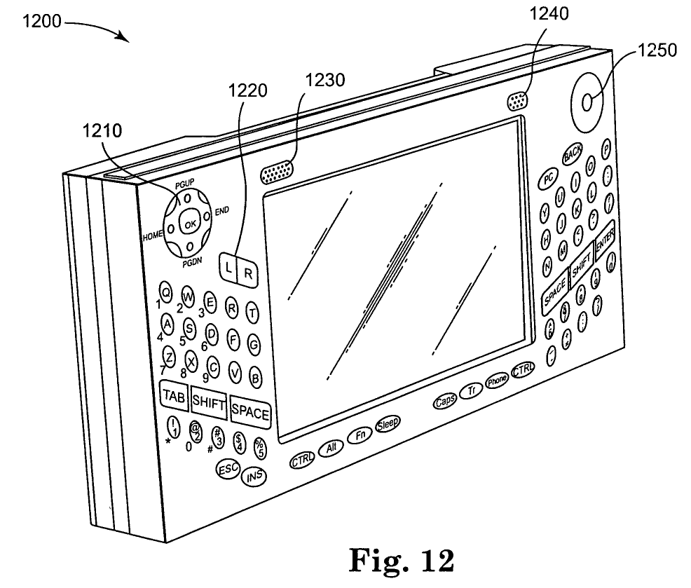

FIG. 12 is an illustration of a variation 1200 of the split onboard keyboard platform configuration of FIG. 4, in which a dual-unit mouse controller is provided. The mouse controller includes a left side control section 1210 with mouse buttons 1220, and a right side control section 1250. The right side control section is a joystick-type controller. Speakers 1230 and 1240 are provided. In other respects, the mobile personal device 1200 is similar to the mobile personal device 400 of FIG. 4.

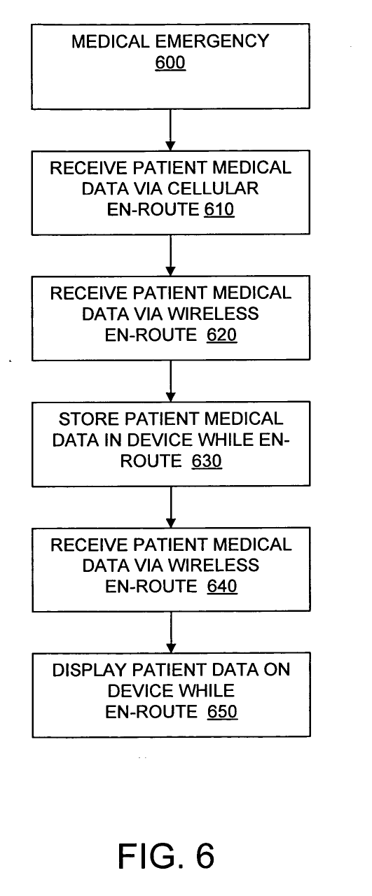

FIG. 5 is an illustration of an alternative platform configuration of a mobile personal device 500 that has a unitary onboard keyboard 532 and an internal GSM antenna 528. Otherwise, the device 500 is substantially identical to the device 400.

In conjunction with a mobile personal device such as any of the devices 100, 400 and 500 described above, many other medical services can be either performed at the point of care or performed on demand as needed. For example, in a battlefield triage scenario, each individual soldier may have medical information pre-stored in an accessible database on his mobile personal device for immediate stat access by corpsmen at the battle scene. Also, with its video capture and transmit capabilities, it may be possible to capture and transmit fingerprint or retinal scan information to identify a downed soldier with no apparent “dog-tag” or other available identification prior to administering on the scene treatment. Another possible use of a mobile personal device in a battlefield scenario is to issue personal mobile personal devices to each military service person prior to deployment such that their medical information is available at the point of care as needed.

Of course, the triage scenario can be expanded to include civilian applications such as emergency medical personnel responding to natural disasters or policemen/firemen responding to injured citizens needing immediate on-site medical attention.

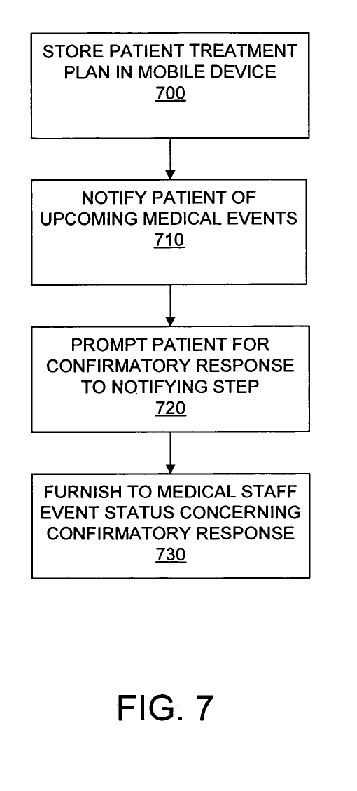

An example of a method for utilizing the mobile personal device to respond to a medical emergency (block 600) is shown in FIG. 6. The mobile personal device may belong and be worn by either the caregiver or the patient. Patient medical data is received in any suitable manner from any relevant source while the caregiver or patient is en route, either by cellular (block 610), or by wireless network (block 620), or a combination thereof. The received data is stored on the caregiver's or the patient's mobile personal device (block 630) for present use and analysis and/or for later use. Additional patient medical data may also be received while the caregiver or patient is en route (block 640), such as, for example, the comments of other team members on the patient's condition or suggestions for testing or treatment. The patient data may be displayed in whole or in part on the mobile personal device while the caregiver or patient is en route. Where the mobile personal device belongs to the caregiver, the caregiver may be en route in any sense of the word, such as, for example, when a physician is going from patient to patient within a hospital or from one ward to another such as the emergency room within the hospital, or when an emergency medical technician is in transit to the patient or is returning from an accident scene with the patient.

The mobile personal device may also have applications in the normal day-to-day operations of the typical doctor's office in Anytown USA. For example, a device carried by a patient may have such information as the patient's medical insurance and medical history information. The physician's scheduling coordinator may with the patient's approval, download the data directly from the patient's mobile personal device via Bluetooth or equivalent transfer techniques. Also, once admitted, the patient can schedule prompts via the patient's mobile personal device to provide a reminder to make upcoming scheduled doctor appointments or to ensure medications are taken on time as prescribed. Furthermore, patient billing and receipt of payment for medical services rendered may be accomplished by the patient utilizing the patient's mobile personal device and its networking capabilities as a financial services tool.

The mobile personal device may also streamline the process, and accuracy thereof, of getting prescriptions filled at the pharmacy. Anyone who has ever viewed their doctor's handwritten prescription has sometime in their life wondered how a pharmacist can decipher the instructions outlining their medications and dosage protocol, never mind the doctor's name from the signature. Utilizing the networking functionality of the mobile personal device, the physician may send the patient's prescription via verbal commands or digital input utilizing the doctor's own personal mobile device with secure log-on access. The transaction may be mediated by a server, so that the prescription may be provided to the pharmacist in any convenient manner, and so that an archival record of the transaction may be preserved for medical history purposes, for prescription adjudication, for insurance reimbursement, and other such purposes. Alternatively, the physician may enter the prescription into the patient's mobile personal device, preferably in a session authenticated by the physician, so that the patient may communicate the prescription to a pharmacist at the patient's convenience, either remotely over a network or during a personal visit to the pharmacy using a short range communications protocol or using a display screen or other output device of the mobile personal device.

The voice activation and verification feature of the mobile personal device, in conjunction with network access, stored historical patient medical data, and near real-time patient medical data, may allow for the device to be used in “sterile only” environments where point-of-care access to near real-time patient information is critical. For example, a surgeon may choose to have their own mobile personal device in close proximity to the operating table (for voice activation) in the event immediate access to either the patient's medical history database is needed or an “over the network” conference call with a remote colleague is needed for advice or a valued second opinion.

The mobile personal device may also make available medical services for the expanding home health care trend, as well as expand the capabilities of hospice care for homebound seniors or disabled patients. The patient's mobile personal device may have vital signs monitoring devices incorporated into its hardware configuration, including wireless capability to communicate with external vital signs sensors. In addition, the homebound patient may use their mobile personal device as a personal medical ledger to keep track of their vital signs, as well as, for example, other physiological parameters important to their health such as blood glucose for the diabetic patient. With pre-authorization, the patient's primary care physician may periodically monitor the patient's medical data between face-to-face office visits by periodically querying the data collected and stored in the patient's personal data base. Even further, the patient's mobile personal device may be pre-programmed or pre-authorized to immediately notify the patient's physician if any of the patient's medical readings exceed predetermined thresholds set either by the patient or their physician, or automatically by algorithm.

The patient's mobile personal device may also have either on-board or accessible through a network, algorithms to process the patients vital and/or metabolic data to perform a wide variety of medical functions. For example, a diabetes patient about to consume a high glucose-generating meal may choose to enter the estimated consumption into their mobile personal device and have an algorithm calculate the suggested insulin dose necessary to properly stabilize the patient's blood sugar level. In a more autonomous environment, the patient's mobile personal device may be in wireless communication with an implantable glucose sensor continuously monitoring the patient's blood glucose, and if the patient's blood glucose level exceeds a predetermined threshold, the mobile personal device may, after calculating the required insulin to stabilize the patient's glucose level via the on-board algorithm, issue a command to the patient's implantable insulin pump (which may also be in wireless communication with the patient's mobile personal device) to deliver same. The above example, highlighting the interaction between the patient's mobile personal device and implantable sensors and devices can be expanded upon to include an array of possibilities. For example, the patient's mobile personal device may be in wireless communication with any number of different types of implantable devices such as pacemakers, defibrillators, nerve and/or muscle stimulators, subcutaneous and transcutaneous drug delivery devices, noninvasive devices such a vital signs monitors, and the like. In some applications such as, for example, the intracardiac device or “ICD” which integrates a pacemaker with built-in defibrillation circuitry, the patient's mobile personal device may be programmed to seek outside authorization (over the network) from the patient's cardiologist before issuing a command to the ICD to modify the pacing or defibrillation function. In this environment, the patient's mobile personal device may serve as a conduit to transfer command-and-control of the patient's ICD to the remote cardiologist.

In addition, the patient's mobile personal device may be programmed to elicit a periodic response from the patient, either verbal or via keystroke, as a means to verify the well being of the patient; for example, verifying that the patient has not become comatose or otherwise incapacitated.

To ensure that the patient's mobile personal device is in communication with only the physiological sensors and dosing units for the patient, which would be important in situations involving crowds and medical facilities, for example, and to prevent tampering or monitoring by unauthorized persons, preferably an exclusive and secure relationship is established between the patient's mobile personal device and the patient's physiological sensors. The relationship may be established in any suitable manner, such as by training, by discovery as under the Bluetooth protocol, by the use of unique device identifiers, by encryption, and so forth. To ensure that the relationship is established under appropriate conditions and only with the patient's physiological sensors and dosing units, the relationship preferably is established during an authenticated session.

The mobile personal device may also have integrated physical devices such as velocity sensors and accelerometers. The accelerometer unit may consist of 3 separate accelerometers oriented orthogonally to independently sense acceleration in the x, y, and z direction respectively. Either the multiple-axes accelerometer or a separate accelerometer may be used to protect the mobile personal device hard drive by sensing when the mobile personal device has been dropped or about to undergo a severe shock. In this event a command is initiated to secure the hard disk to prevent damage. Given this, the patient may attach their mobile personal device to their belt similar to the way calculators and cell phones are currently used. Then, if the patient were to undergo a sudden acceleration or de-acceleration such as by falling down a flight of stairs or hitting a tree in a high speed car wreck, the mobile personal device may be pre-programmed to autonomously issue a “911” call on the user's behalf if the acceleration/de-acceleration exceeds a pre-determined threshold, supplying approximate patient location via its GPS capabilities. In addition, the patient's mobile personal device may be programmed to check with the patient before issuing the emergency notification. If a suitable response is received from the patient, either verbal or via keystroke, to verify the well being of the patient, the emergency notification is not sent.

In all of the examples listed above, access to the owner's mobile personal device, and in particular to its database and command-and-control functions, preferably is granted only upon authentication of the user's identity through positive proof-of-identity, with different persons potentially having different privileges. Access by the owner may be authorized by voice, fingerprint, retinal scan, or a combination of these to gain preferably unlimited access and control. The owner may establish a log-on protocol for others to access only particular information and perform only particular functions using the owner's mobile personal device. Where the owner is a patient, for example, the owner may establish a hierarchy for caregivers based on the caregiver's need to know and relationship to the owner. In the situation presented above wherein the patient does not respond to the periodic query from their mobile personal device, in addition to initiating the “911” call, the protocol may not only allow the physician unlimited access to the patient's medical database on the patient's mobile personal device, but may simultaneously prompt the physician and give access to the physician to read and review other related files such as electronic documents pertaining to “My care and treatment if clinically brain dead and issuance of a Due Not Resuscitate (DNR) form on my behalf.” This type of access may be coined “situational access” and can have many layers of detail. To actually gain access to these files (similar to the patient) the physician may have to provide voice, fingerprint and/or retinal data, either directly to the patient's mobile personal device or over the network. A particularly secure technique suitable for sensitive situations such as the DNR form, the physician may be required to enter suitable biometrics data such as voice, fingerprint, retinal scan, and so forth directly into the patient's mobile personal device to gain access.

Since a patient's last wishes may be challenged by a number of different entities, both within family and by outside governmental bodies, the networking capabilities of the patient's mobile personal device may be used to adjudicate the patient's last wishes. The patient's mobile personal device may automatically or manually store a duplicate copy of a DNR type last wishes on a remote networked server. The duplicate copy may be validated with the patient's own biometrics data so as to serve as a credible second source to verify the patient's terminal care request. In less extreme cases, the patient's lawyer may be given routine first-level access to “read-only” legal documents stored in the patient's mobile personal device via voice recognition only, and may require multiple proof-of-identity qualifiers (e.g., voice and fingerprint) including patient keystroke or verbal command approval to edit existing documents or generate new entries.

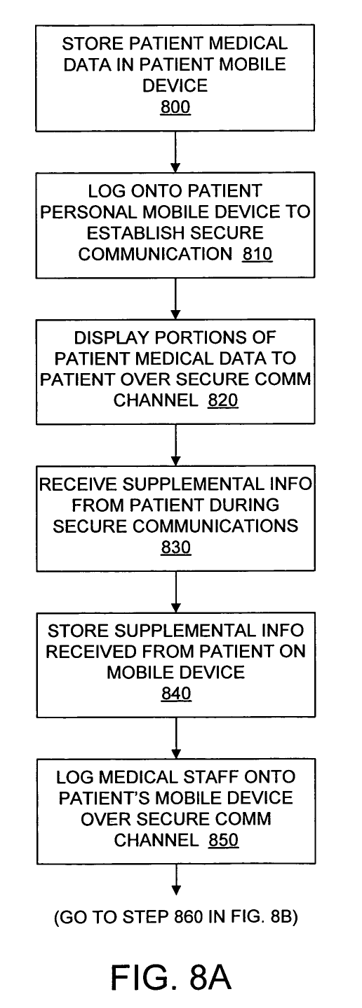

One example of a method for interacting with a patient who utilizes the mobile personal device is shown in FIG. 7. A patient's treatment plan is stored in the patient's mobile personal device (block 700). The patient's mobile personal device may then notify the patient of upcoming medical events (block 710), prompt the patient for confirmatory responses to the notification (block 720), and furnish to a caregiver information concerning the patient's responses or lack thereof (block 730).

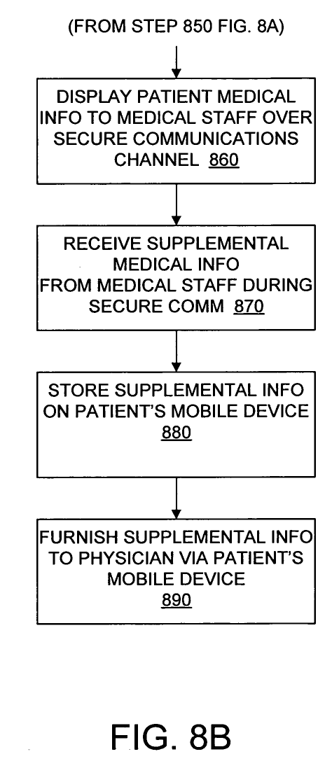

Another example of a method for interacting with a patient who utilizes the mobile personal device is shown in FIG. 8A and FIG. 8B. Patient medical data is stored in the patient's mobile personal device (block 800). The patient may log onto the patient's mobile personal device using secure communications (block 810) over which the patient's medical data may be viewed by the patient (block 820). Additionally, supplemental information may be received from the patient using the secure communications (block 830) and may also be stored on the patient's mobile personal device (block 840). Medical personnel may also log onto the patient's mobile personal device using secure communications (block 850) over which the patient's medical data may be viewed by the medical personnel (block 860). Additionally, supplemental information may be received from the medical personnel using the secure communications (block 870) and may also be stored on the patient's mobile personal device (block 880). The supplemental information may be furnished to the physician from the patient's mobile personal device (block 890), with or without other patient medical data.

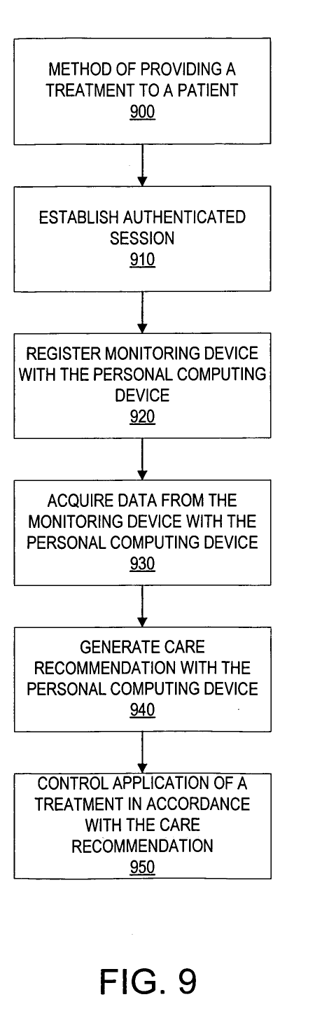

FIG. 9 is a flowchart of a method 900 of providing a treatment to a patient having a mobile personal computing device. An authenticated session is established (block 910), and a patient-associated physiological monitoring device is registered with the personal computing device during the authenticated session (block 920). Patient physiological data is securely acquired from the patient-associated physiological monitoring device with the personal computing device (block 930). The patient physiological data is analyzed with the personal computing device to generate a patient care recommendation (block 940). The application of a treatment is controlled in accordance with the patient care recommendation (block 950).

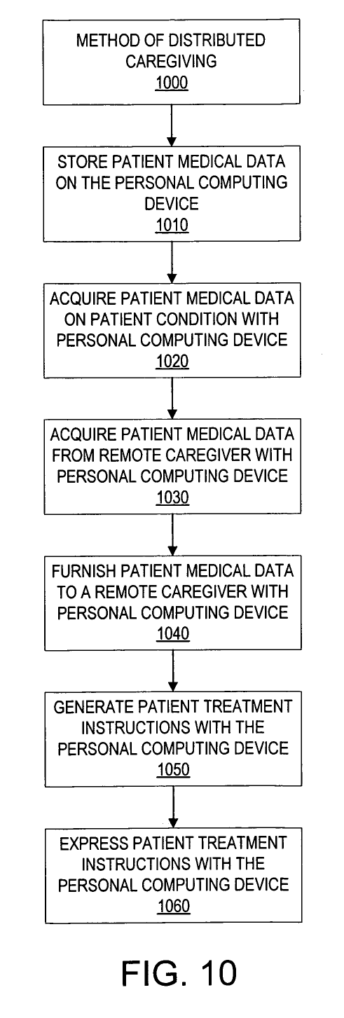

FIG. 10 is a flowchart of a method 1000 of distributed caregiving to a patient having a mobile personal computing and communications device at a point-of-care. Patient medical data is stored in the personal computing and communications device at the point-of-care (block 1010). Patient medical data regarding a current condition of the patient is acquired with the personal computing and communications device at the point-of-care (block 1020). Patient medical data is acquired from at least one remote caregiver in an authenticated session using the communications capability of the personal computing and communications device, at the point-of-care (block 1030). Selected items from the patient medical data are furnished to at least one remote caregiver in an authenticated session using the communications capability of the personal computing and communications device (block 1040). Patient treatment instructions for the current condition are generated with the personal computing and communications device from the patient medical data (block 1050). The patient treatment instructions are expressed with the personal computing and communications device at the point-of care (block 1060).

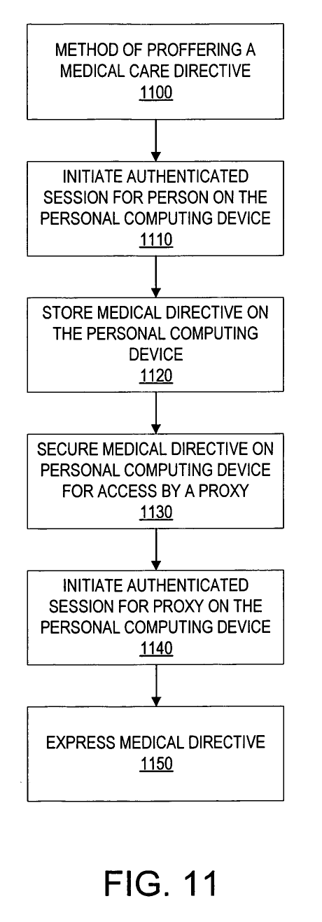

FIG. 11 is a flowchart of a method 1100 of proffering an authenticated medical directive of a person. A first authenticated session is initiated on a personal computing device with a biometric of the person (block 1110). A medical directive is stored on the personal computing device during the patient-authenticated session (block 1120). During the patient-authenticated session, the stored medical directive is secured for access by a proxy, the proxy being identified by a biometric of the proxy (block 1130). A second authenticated session is initiated on the personal computing device with the biometric of the proxy (block 1140). The secured medical directive is expressed during the second authenticated session (block 1150).

Following is a further description of various apparatus, methods and systems for mobile medical services.

There is a need within the medical community for near-real-time point-of-care accurate medical information and on demand access to healthcare professionals on an as needed stat basis. This may be done with a communication system for examination and treatment of a patient by medical personnel at a remote location utilizing a handheld mobile personal computing and communications device with integrated phone and secure wireless communications. The patient's medical records are accessible utilizing the communications capability of the mobile personal device while medical personnel are en-route to the patient. This may be done in accordance with a method for examining and treating a patient by medical personnel at a remote location utilizing a handheld mobile personal device computer with integrated phone and secure wireless communications. The patient's medical records are accessed utilizing the communications capability of the mobile personal device while medical personnel are en-route to the patient. A further method is for initiating an emergency response by providing emergency medical responders with a handheld mobile personal computing and communications device with integrated phone and secure wireless communications. The mobile personal device provides secure communications for transmitting and receiving voice, data, and video images as well as initiating the emergency response command.

A method for ensuring patient's compliance with a prescribed medical treatment plan, including a prescription protocol, may comprise utilizing a handheld mobile personal computing and communications device with integrated phone and secure wireless communications. The device may remind the patient of upcoming medical events such as dosing and appointments, and may prompt the patient for a confirmatory response.

A method for a medical worker to prepare for a patient visit while the medical worker is in route to the patient may comprise receiving notification of a need for the patient visit on a mobile personal device having a size, weight and form factor suitable for mobile use by the medical worker, the mobile personal device having a cellular communications capability and a broadband wireless communications capability contained within the unit, and the notification being received via one of the cellular communications capability and the broadband wireless communications capability. First medical information is acquired relating to the patient via the cellular communications capability while the device is in route to the patient with the medical worker. Second medical information is acquired relating to the patient via the broadband wireless communications capability while the device is in route to the patient with the medical worker. The second medical information is stored on a storage medium while the device is in route to the patient with the medical worker, the storage medium being included in the device. The medical information is displayed on a display screen while the device is in route to the patient with the medical worker, the display screen being included in the device.

The medical worker preparation method may further comprise furnishing a network comprising a plurality of separate wireless local area networks; and roaming among the wireless local area networks with the device while the device is in route to the patient with the medical worker, the device and the network being cooperative to achieve a seamless network connection with session persistence. The second medical information acquiring step comprises acquiring the second medical information via the broadband wireless communications capability and the wireless area networks while the device is in route to the patient with the medical worker.

Alternatively, the medical worker preparation method may further comprise logging the medical worker onto the device by a secure logon procedure executed by the device, prior to the medical information displaying step.

Alternatively, the medical worker preparation method may further comprise acquiring an indication of position of the patient; acquiring an indication of position of the device while the device is in route to the patient with the medical worker with a global positioning system capability, the global positioning system capability being included in the device; generating travel directions in the device based on the indication of position of the device and the indication of position of the patient, the device having a processor, stored mapping data, and stored executable code therefor; and furnishing the travel directions to the medical worker from the device.

Alternatively, the medical worker preparation method may further comprise acquiring an indication of position of the device while the device is in route to the patient with the medical worker with a global positioning system capability, the global positioning system capability being included in the device; and furnishing the indication of position of the device to other medical workers en route to the patient, via one of the cellular communications capability and the broadband wireless communications capability.

Alternatively where the second information comprises patient biometrics identity information, the medical worker preparation method may further comprise acquiring a biometrics identity measurement from the patient; and comparing the biometrics identity measurement with the biometrics identity information in the device to confirm patient identity, the device having a processor and stored executable code therefor. In a variation, the biometrics identity measurement acquiring step may comprise acquiring at least one of a fingerprint image with a scanner included in the device, and a retinal image with a camera included in the device.

Alternatively, the medical worker preparation method may further comprise acquiring a biometrics identity measurement from the patient; transmitting the biometrics identity measurement to a remote server via one of the cellular communications capability and the broadband wireless communications capability to identify the patient; and receiving information on the patient's identity from the remote server via one of the cellular communications capability and the broadband wireless communications capability. In a variation, the biometrics identity measurement acquiring step may comprise acquiring at least one of a fingerprint image with a scanner included in the device, and a retinal image with a camera included in the device.

Alternatively, the medical worker preparation method may further comprise furnishing a network comprising a plurality of separate wireless local area networks; roaming among the wireless local area networks with the device while the device is in route to the patient with the medical worker, the device and the network being cooperative to achieve a seamless network connection with session persistence; and maintaining a teleconference regarding the patient between the medical worker and a remote party via the broadband wireless communications capability and the wireless area networks while the device is in route to the patient with the medical worker, the device having a camera, a processor, and stored executable code therefor.

Alternatively, the medical worker preparation method may further comprise acquiring a biometrics measurement from the patient; generating an indication of a physiological state of the patient from the biometrics measurement, the device having a processor and stored executable code therefor; and displaying the physiological state indication on the display screen. In one variation, the biometrics measurement acquiring step may comprise acquiring at least one of a first measurement from a blood pressure sensor included in the device; a second measurement from a temperature sensor included in the device; a third measurement from a heart rate monitor included in the device; a fourth measurement from a blood oxygen saturation sensor included in the device; a fifth measurement from a blood glucose sensor included in the device; and a sixth measurement from an electrocardiograph included in the device. In another variation, the biometrics measurement acquiring step may comprise acquiring at least one of a first measurement from a blood pressure sensor remote to the device via a wireless personal area network capability included in the device; a second measurement from a temperature sensor remote to the device via the wireless personal area network capability; a third measurement from a heart rate monitor remote to the device via the wireless personal area network capability; a fourth measurement from a blood oxygen saturation sensor remote to the device via the wireless personal area network capability; a fifth measurement from a blood glucose sensor remote to the device via the wireless personal area network capability; and a sixth measurement from an electrocardiograph remote to the device via the wireless personal area network capability.

Alternatively in the medical worker preparation method, the second medical information acquiring step may comprise acquiring at least one of a medical history, an x-ray record, a dental record, blood chemistry data, laboratory data, prescription history, and allergic reaction information.

Alternatively in the medical worker preparation method, the first medical information acquiring step may comprise engaging in conversation using a hands-free capability associated with the cellular communications capability while the device is in route to the patient with the medical worker.

Alternatively, the medical worker preparation method may further comprise controlling the device orally while the device is in route to the patient with the medical worker, a processor, microphone, and executable speech recognition code being included in the device therefor.

Alternatively in the medical worker preparation method, the notification receiving step may comprises establishing a priority table to designate an entity group having emergency medical response priority; monitoring the cellular communications capability and the broadband wireless communications capability for an emergency communication from any member of the emergency medical response priority entity group, in accordance with the priority table; and informing the medical worker of the emergency communication. The priority table may be stored on the storage medium and the device may further including executable code stored on the storage medium and a processor for the establishing, monitoring and informing steps.

Alternatively in the medical worker preparation method, the medical worker may be en route to the patient along a route at least in major part remote from a medical facility.

Alternatively in the medical worker preparation method, the medical worker may be en route to the patient along a route entirely within a medical facility.

Alternatively where the second medical information comprises an identification of next of kin, the medical worker preparation method may further comprise contacting the next of kin via one of the cellular communications capability and the broadband wireless communications capability to obtain authorization to treat the patient.

A medical worker also may prepare for a patient visit while the medical worker is in route to the patient in accordance with a method that comprises receiving notification of a need for the patient visit on a mobile personal device having a size, weight and form factor suitable for mobile use by the medical worker, the mobile personal device having a cellular communications capability and a broadband wireless communications capability contained within the unit, and the notification being received via one of the cellular communications capability and the broadband wireless communications capability; acquiring first medical information relating to the patient via the cellular communications capability while the device is in route to the patient with the medical worker; acquiring second medical information relating to the patient via the broadband wireless communications capability while the device is in route to the patient with the medical worker; storing the second medical information on a storage medium while the device is in route to the patient with the medical worker, the storage medium being included in the device; and displaying the medical information on a display screen while the device is in route to the patient with the medical worker, the display screen being included in the device.

A method for facilitating compliance with a patient treatment plan may comprise storing the patient treatment plan on a storage medium of a mobile personal device having a size, weight and form factor suitable for mobile use by the patient; notifying the patient of an upcoming medical event from the device based on the stored patient treatment plan, the device having a processor and executable code therefor, the executable code being stored on the storage medium; prompting the patient for a confirmatory response to the notifying step acknowledging the upcoming medical event; furnishing to a medical worker from the device medical event status information pertaining to whether the confirmatory response was made in response to the prompting step, the device having a cellular communications capability and a broadband wireless communications capability contained within the unit, and the medical event status information being furnished via one of the cellular communications capability and the broadband wireless communications capability.

The compliance facilitating method may further comprise updating the patient treatment plan on the device from a physician facility via the broadband wireless capability. In a variation, changed portions of the patient treatment plan resulting from the updating step may be displayed to the patient on a display screen, the device including the display screen.

Alternatively, the compliance facilitating method may further comprise storing a message from the patient on the storage medium; and furnishing the patient message to a medical worker from the device via one of the cellular communications capability and the broadband wireless communications capability.

Alternatively where the upcoming medical event is a physician appointment, the compliance facilitating method may further comprise canceling the physician appointment in response to the medical event status information.

Alternatively, the compliance facilitating method may further comprise dispatching emergency response personnel in response to the medical event status information.

Alternatively where the upcoming medical event is a medication dosing event and the confirmatory response comprises acknowledgment that a prescribed dosage was taken at a proper time, the compliance facilitating method may further comprise storing compliance with the patient treatment plan on the storage medium.

Alternatively where the upcoming medical event is a medication dosing event and the confirmatory response is absent or comprises an indication of an improper dosage or an improper time, the compliance facilitating method may further comprise storing an indication of non-compliance with the patient treatment plan on the storage medium; and furnishing the indication of non-compliance to a medical worker from the device via one of the cellular communications capability and the broadband wireless communications capability.

Compliance with a patient treatment plan may also be facilitated in a method comprising storing the patient treatment plan on a storage medium of a mobile personal device having a size, weight and form factor suitable for mobile use by the patient; notifying the patient of an upcoming medical event from the device based on the stored patient treatment plan, the device having a processor and executable code therefor, the executable code being stored on the storage medium; prompting the patient for a confirmatory response to the notifying step acknowledging the upcoming medical event; and furnishing to a medical worker from the device medical event status information pertaining to whether the confirmatory response was made in response to the prompting step, the device having a cellular communications capability and a broadband wireless communications capability contained within the unit, and the medical event status information being furnished via one of the cellular communications capability and the broadband wireless communications capability.

A method for facilitating care of a patient may comprise storing medical information for the patient on a storage medium of a mobile personal device having a size, weight and form factor suitable for mobile use; logging the patient onto the device by a secure logon procedure executed by the device to establish a secure patient session; displaying portions of the patient medical information to the patient on a display screen included in the device during the secure patient session; receiving supplemental medical information from the patient during the secure patient session; storing the supplemental medical information from the patient on the storage medium; logging a medical worker onto the device by a secure logon procedure executed by the device to establish a secure medical worker session; displaying portions of the patient medical information to the medical worker on the display screen during the secure patient session; receiving supplemental medical information from the medical worker during the secure patient session; storing the supplemental medical information from the medical worker on the storage medium; and furnishing the supplemental medical information from the patient and the supplemental medical information from the medical worker to a physician from the device via one of the cellular communications capability and the broadband wireless communications capability.

The care facilitating method may further comprise incorporating a date-time stamp into the supplemental medical information from the patient prior to the step of storing the supplemental medical information from the patient on the storage medium; and incorporating a date-time stamp into the supplemental medical information from the medical worker prior to the step of storing the supplemental medical information from the medical worker on the storage medium.

Alternatively, the care facilitating method may further comprise acquiring a biometrics measurement from the patient during at least one of the secure patient session and the secure medical worker session; generating an indication of a physiological state of the patient from the biometrics measurement, the device having a processor and stored executable code therefor; and displaying the physiological state indication on the display screen. One variation may further comprise storing the physiological state on the storage medium; and furnishing the physiological state to a physician from the device via one of the cellular communications capability and the broadband wireless communications capability. In a second variation, the biometrics measurement acquiring step may comprise acquiring at least one of a first measurement from a blood pressure sensor included in the device; a second measurement from a temperature sensor included in the device; a third measurement from a heart rate monitor included in the device; a fourth measurement from a blood oxygen saturation sensor included in the device; a fifth measurement from a blood glucose sensor included in the device; and a sixth measurement from an electrocardiograph included in the device.

Alternatively, the care facilitating method may further comprise evaluating with the device the supplemental medical information from the patient and the supplemental medical information from the medical worker to detect an abnormality, the device including a processor and executable code therefor; and furnishing notification of the abnormality to a physician from the device via one of the cellular communications capability and the broadband wireless communications capability.

Alternatively, the care facilitating method may further comprise acquiring physician signature information from a prescription using a sensor included in the device during at least one of the secure patient session and the secure medical worker session; comparing the physician signature information with a physician signature sample to determine an indication of authenticity; and displaying the indication of authenticity on the display screen.

Alternatively, the care facilitating method may further comprise acquiring prescription identifying information from a prescription dispenser label using a sensor included in the device during at least one of the secure patient session and the secure medical worker session; acquiring with the device original prescription information via one of the cellular communications capability and the broadband wireless communications capability; generating with the device an indication of prescription accuracy from a comparison of the prescription identifying information with the original prescription information, the device having a processor and stored executable code therefor; and displaying the indication of prescription accuracy on the display screen.

Care of a patient may also be facilitated in a method that comprises storing medical information for the patient on a storage medium of a mobile personal device having a size, weight and form factor suitable for mobile use; logging the patient onto the device by a secure logon procedure executed by the device to establish a secure patient session; displaying portions of the patient medical information to the patient on a display screen included in the device during the secure patient session; receiving supplemental medical information from the patient during the secure patient session; storing the supplemental medical information from the patient on the storage medium; logging a medical worker onto the device by a secure logon procedure executed by the device to establish a secure medical worker session; displaying portions of the patient medical information to the medical worker on the display screen during the secure patient session; receiving supplemental medical information from the medical worker during the secure patient session; storing the supplemental medical information from the medical worker on the storage medium; and furnishing the supplemental medical information from the patient and the supplemental medical information from the medical worker to a physician from the device via one of the cellular communications capability and the broadband wireless communications capability.

An apparatus useful in the examination and treatment of a patient by a medical industry user may comprise a unit having a size, weight and form factor suitable for mobile use by the user; a processor capability contained within the unit and having sufficient processing power for executing computer programs that are designed for personal computers; a cellular communications capability contained within the unit; a broadband wireless communications capability contained within the unit; a wireless personal area network capability contained within the unit; a security capability contained within the unit and functionally integrated with the processor capability, the cellular communications capability; and the broadband wireless capability for providing a secure login for the unit and secure wireless communications; a global positioning system capability functionally integrated with the processor capability; an optical scanner capability functionally integrated with the processor capability; a camera capability functionally integrated with the processor capability and the cellular communications capability; and a storage medium readable by the processor subsystem and comprising executable code.

A communications system for examination and treatment of a patient by medical personnel at a location remote from a medical facility may comprise a hand-held personal computer with integrated cellular telephone, including a computer processor capable of executing a plurality of application programs, including email, personal productivity programs, internet programs and other conventional personal computer programs; at least one integrated secure wireless communications network; at least one integrated wireless communications network interface integrated within the hand-held personal computer; and at least one medical facility, the facility storing the medical records of the patient at least in part in electronic form, and wherein the medical records are provided over the wireless communications network to the medical personnel via the hand-held personal computer while en route to the patient.

The communications system may further comprise an optical scanner for obtaining a fingerprint image of the patient for identification confirmation of the patient.

Alternatively, the communications system may further comprise a retinal scanner for obtaining an image of the patient's retina for identification confirmation of the patient.

Alternatively, the communications system may further comprise a blood pressure sensor, integrated into the system; a temperature sensor integrated into the system; a heart rate monitor integrated into the system; a blood oxygen saturation sensor integrated into the system; a blood glucose sensor integrated into the system; and an electrocardiograph integrated into the system.

Alternatively, the communications system may further comprise a digital camera, the camera capable of transmitting and receiving video images to allow teleconferencing with personnel at the medical facility from the remote location regarding the patient's condition.

Alternatively, the communications system may further comprise a video display, wherein video images received from the medical facility may be viewed by the medical personnel. In a variation, the video images received and viewed comprise the patient's medical history, x-rays, dental records and the like.

A method for examining and treating a patient by medical personnel at a location remote from a medical facility may comprise providing a hand-held personal computer with integrated cellular telephone, including a computer processor capable of executing a plurality of application programs, including email, personal productivity programs, internet programs and other conventional personal computer programs; providing at least one integrated secure wireless communications network; providing at least one secure wireless communications network interface integrated within the hand-held personal computer; and downloading relevant patient information while the medical personnel is en route to the patient.

In the patient examining and treating method, the patient information downloaded may comprise the patient's medical history, known allergies, x-rays and the like.

The patient examining and treating method may further comprise video teleconferencing with medical experts concerning the patient's condition using the hand-held personal computer and integrated telephone. In a variation, the method may further comprise video teleconferencing with medical personnel while en route to the patient.

Alternatively, the patient examining and treating method may further comprise obtaining a fingerprint image of the patient using a scanner integrated in the hand-held computer; transmitting the fingerprint image to a central database for correlation; and identifying the patient based on a fingerprint match. In a variation, the method may further comprise scanning the patient's retina using a retinal scanner integrated in the hand-held computer; obtaining an image of the patient's retina for identification confirmation of the patient; transmitting the retinal image to a central database for correlation; and identifying the patient based on a retinal image match.

Alternatively, the patient examining and treating method may further comprise providing the medical personnel with hands-free communication with medical personnel at the medical facility via the hand-held personnel computer with integrated phone.

Alternatively, the patient examining and treating method may further comprise providing voice-activated, speech recognition communications capability to the hand-held personal computer.

A method for initiating an emergency response may comprise providing emergency medical responders with a hand-held personal computer with an integrated telephone, including a computer processor capable of executing a plurality of application programs, including email, personal productivity programs, internet programs and other conventional personal computer programs; providing an integrated secure communications network for transmitting and receiving voice, data and, video and image communications using the hand-held personal computer with integrated telephone; and initiating an emergency medical response over the integrated secure communications network.

The emergency response initiation method may further comprise accessing a patient's medical information using the hand-held personal computer and integrated telephone. In a variation, the patient's medical information may be accessed while en-route to the patient. In another variation, the patient's medical information may comprise the patient's medical history, blood type, lab results, current medications the patient may be taking, and the patient's known allergic reactions.

Alternatively, the emergency response initiation method may further comprise video teleconferencing with medical experts concerning the patient's condition using the hand-held personal computer and integrated telephone.

Alternatively, the emergency response initiation method may comprise initiating the emergency medical response at a location remote from a medical facility.

Alternatively, the emergency response initiation method may comprise initiating the emergency medical response within a medical facility.

Alternatively, the emergency response initiation method may further comprise using the hand-held personal computer and integrated telephone to obtain authorization to treat the patient from the patient's next of kin.

Alternatively, the emergency response initiation method may further comprise obtaining a fingerprint image of the patient using a scanner integrated in the hand-held computer; transmitting the fingerprint image to a central database for correlation; and identifying the patient based on a fingerprint match.

Alternatively, the emergency response initiation method may further comprise scanning the patient's retina using a retinal scanner integrated in the hand-held computer; obtaining an image of the patient's retina for identification confirmation of the patient; transmitting the retinal image to a central database for correlation; and identifying the patient based on a retinal image match.

A method for ensuring compliance with medical patient treatment plans and prescriptions using a communications device may comprise providing the patient with a medical treatment plan; providing the patient with a hand-held personal computer with an integrated telephone, including a computer processor capable of executing a plurality of application programs, including email, personal productivity programs, internet programs and other conventional personal computer programs; providing at least one wireless network communication interface on the hand-held personal computer; providing at least one wireless network that the hand-held personal computer may interface with; providing the patient with at least one reminder of an upcoming medical appointment; prompting the patient for confirmatory response from patient via the hand-held personal computer; and confirming the appointment when the patient responds affirmatively.

The compliance ensuring method may further comprise canceling the appointment if the patient responds negatively to the confirmatory prompt.

Alternatively, the compliance ensuring method may further comprise notifying the patient's physician if the patient fails to respond to the confirmatory prompt.

Alternatively, the compliance ensuring method may further comprise reminding the patient of the type, dosage and frequency of medication prescribed according to the medical treatment plan; requesting confirmation from the patient that the proper dosage of medication was taken at the appropriate time; clearing the reminder if the patient confirms; notifying the patient's physician if the patient confirms with incorrect medication or dosage; and notifying the patient's physician if the patient fails to confirm.

Alternatively, where the medical treatment plan includes home health care, the compliance ensuring method may further comprise allowing the patient to enter personal, date-time stamped medical information; and providing the patient's physician access to the patient's personal, date-time stamped medical information for review and evaluation. In a variation, at least one home health care medical device may be integrated into the hand-held computer. Examples of suitable home health care medical devices include a blood pressure sensor, temperature sensor integrated into the system, a heart rate monitor, a blood oxygen saturation sensor, a blood glucose sensor, and an electrocardiograph.

Alternatively, where the medical treatment plan includes home health care, the compliance ensuring method may further comprise allowing the patient to enter personal, date-time stamped medical information; comparing the patient's medical information with a predetermined normal range of values; and notifying the patient and the patient's physician if the medical information is determined to be outside the predetermined normal range of values.

Alternatively, the compliance ensuring method may further comprise scanning written prescriptions; and determining the authenticity of the prescribing doctor's signature to ensure integrity and validity of the prescription. In a variation, the scanning is performed by the pharmacist filling the scanned prescription. In another variation, the prescribing Doctor's signature on the prescription may be correlated with a sample signature specimen to determine the authenticity of the prescription signature.

An additional method for facilitating care of a patient may comprise storing medical information for the patient on a storage medium of a mobile personal device having a size, weight and form factor suitable for mobile use; logging the patient onto the device by a secure logon procedure executed by the device to establish a secure patient session; displaying portions of the patient medical information to the patient on a display screen included in the device during the secure patient session; receiving supplemental medical information from the patient during the secure patient session; storing the supplemental medical information from the patient on the storage medium; and furnishing the supplemental medical information from the patient to a physician from the device via one of the cellular communications capability and the broadband wireless communications capability.

The additional care facilitating method may further comprise incorporating a date-time stamp into the supplemental medical information from the patient prior to the step of storing the supplemental medical information from the patient on the storage medium.

Alternatively, the additional care facilitating method may further comprise acquiring a biometrics measurement from the patient during the secure patient session; generating an indication of a physiological state of the patient from the biometrics measurement, the device having a processor and stored executable code therefor; and displaying the physiological state indication on the display screen.

Alternatively, the additional care facilitating method may further comprise acquiring a biometrics measurement from the patient during the secure patient session; generating an indication of a physiological state of the patient from the biometrics measurement, the device having a processor and stored executable code therefor; storing the physiological state on the storage medium; and furnishing the physiological state to a physician from the device via one of the cellular communications capability and the broadband wireless communications capability.

It will therefore be appreciated that the description of the invention including its applications and advantages as set forth herein is illustrative and is not intended to limit the scope of the invention as set forth in the claims. Variations and modifications of the embodiments disclosed herein are possible, and practical alternatives to and equivalents of the various elements of the embodiments would be understood to those of ordinary skill in the art upon study of this patent document. These and other variations, modifications, alternatives and equivalents may be practiced without departing from the scope and spirit of the invention.

1. A method of providing a treatment to a patient having a mobile personal computing device with a size, weight and form factor suitable for mobile use by the patient, comprising:
registering a patient-associated physiological monitoring device with the personal computing device during an authenticated session;
securely acquiring patient physiological data from the patient-associated physiological monitoring device with the personal computing device;
analyzing the patient physiological data with the personal computing device to generate a patient care recommendation; and
controlling the application of a treatment in accordance with the patient care recommendation.

2. The method of claim 1 wherein the registering step comprises:
initiating the authenticated session with a biometric of the patient; and
establishing an exclusive relationship between the patient-associated physiological monitoring device and the personal computing device.

3. The method of claim 1 further comprising:
registering an additional patient-associated physiological monitoring device with the personal computing device in an authenticated session;
wherein the acquiring step further comprises securely acquiring the patient physiological data from both the patient-associated physiological monitoring device and the additional patient-associated physiological monitoring device with the personal computing device.

4. The method of claim 3 wherein the patient-associated physiological monitoring device and the additional patient-associated physiological monitoring device registering steps are completed in one authenticated session.

5. The method of claim 3 wherein the patient-associated physiological monitoring device and the additional patient-associated physiological monitoring device registering steps are completed in respective different authenticated sessions.

6. The method of claim 1 wherein the patient-associated physiological monitoring device is a subcutaneous device.

7. The method of claim 1 wherein the patient-associated physiological monitoring device is a transcutaneous device.

8. The method of claim 1 wherein the patient-associated physiological monitoring device is an implanted device.

9. The method of claim 1 wherein the patient-associated physiological monitoring device is a noninvasive sensor.

10. The method of claim 1 wherein the analyzing step comprises:
establishing an algorithm in the personal computing device for deriving care recommendations from physiological data; and
applying the algorithm to the patient physiological data to generate the patient care recommendation.

11. The method of claim 10 further comprising:
establishing patient medical data in addition to the patient physiological data in the personal computing device;
wherein the algorithm applying step further comprises applying the algorithm to the patient medical data as well as the patient physiological data to generate the patient care recommendation.

12. The method of claim 11 further comprising augmenting the patient medical data with the patient physiological data in the personal computing device in near-real time with acquisition of the patient physiological data.

13. The method of claim 1 wherein:
the patient care recommendation comprises a dosing command; and
the controlling step further comprises communicating the dosing command from the personal computing device to a dosing device associated with the patient.

14. The method of claim 13 wherein the dosing device is an implantable insulin pump.

15. The method of claim 13 wherein the dosing device is an implantable nerve stimulator.

16. The method of claim 13 wherein the dosing device is an implantable cardiac device.

17. The method of claim 13 wherein the dosing device is an externally applied defibrillator.

18. The method of claim 1 wherein:
the patient care recommendation comprises a dosing command; and
the controlling step further comprises:
presenting the dosing command to a decisionmaker; and
communicating the dosing command from the personal computing device to a dosing device associated with the patient upon approval of the dosing command by the decisionmaker.

19. The method of claim 18 wherein the decisionmaker is the patient.

20. The method of claim 18 wherein the decisionmaker is a physician.

21. The method of claim 20 wherein:
the personal computing device comprises a wireless communications capability;
the physician is remote from the patient;
the presenting step comprises transmitting the patient care recommendation to the physician via the communications capability of the personal computing device; and
the communicating step comprises receiving approval of the patient care recommendation from the physician via the communications capability of the personal computing device.

22. A mobile personal computing device for providing a treatment to a patient having a patient-associated physiological monitoring device, comprising:
means for initiating a patient-authenticated session;
means for registering the patient-associated physiological monitoring device with the personal computing device during the patient-authenticated session;
means for securely acquiring patient physiological data from the patient-associated physiological monitoring device with the personal computing device;
means for analyzing the patient physiological data with the personal computing device to generate a patient care recommendation; and
means for controlling the application of a treatment in accordance with the patient care recommendation;
wherein the mobile personal computing device is of a size, weight and form factor suitable for mobile use by the patient.

23. The mobile personal computing device of claim 22 wherein:
the patient care recommendation comprises a dosing command; and
the controlling means further comprises:
means for presenting the dosing command to a decisionmaker; and
means for communicating the dosing command from the personal computing device to a dosing device associated with the patient upon approval of the dosing command by the decisionmaker.

24. The mobile personal computing device of claim 23 further comprising:
wireless communications means;
wherein the presenting means comprises means for transmitting the patient care recommendation to the decisionmaker via the communications means, the decisionmaker being remote from the patient; and
wherein the communicating means comprises means for receiving approval of the patient care recommendation from the decisionmaker via the communications means.

25. A method of distributed caregiving to a patient having a mobile personal computing and communications device at a point-of-care, the personal computing and communications device having a wireless communications capability, and further having a size, weight and form factor suitable for mobile use by the patient, comprising:
storing first patient medical data in the personal computing and communications device at the point-of-care;
acquiring second patient medical data regarding a current condition of the patient with the personal computing and communications device at the point-of-care;
acquiring third patient medical data from at least one remote caregiver in an authenticated session using the communications capability of the personal computing and communications device, at the point-of-care;
furnishing selected items from at least one of the first, second and third patient medical data to at least one remote caregiver in an authenticated session using the communications capability of the personal computing and communications device;
generating patient treatment instructions for the current condition with the personal computing and communications device from the third patient medical data; and
expressing the patient treatment instructions with the personal computing and communications device at the point-of care.

26. The method of claim 25 wherein the second patient medical data acquiring step comprises acquiring patient physiological data from a patient-associated physiological monitoring device in at least near real-time with the current condition.

27. The method of claim 25 wherein the second patient medical data acquiring step comprises acquiring patient medical data from a caregiver located at the point-of-care via a user interface of the personal computing and communications device.

28. The method of claim 25 wherein the second patient medical data acquiring step comprises acquiring patient medical data from the patient via a user interface of the personal computing and communications device.

29. The method of claim 25 wherein:
the caregiver is a physician; and
the third patient medical data is physician diagnostic data.

30. The method of claim 25 wherein the third patient medical data is revised dosing data.

31. The method of claim 25 wherein the third patient medical data acquiring step and the furnishing step involve a plurality of caregivers, each furnishing respective third patient medical data to the other caregivers via the personal computing and communications device, and each receiving respective selected items from at least one of the first, second and third patient medical data from the other caregivers via the personal computing and communications device.

32. The method of claim 31 wherein the plurality of caregivers comprise at least one physician.

33. The method of claim 32 wherein the plurality of caregivers comprise at least one home care nurse.

34. The method of claim 32 wherein the plurality of caregivers comprise at least one emergency medical technician.

35. The method of claim 25 wherein the generating step comprises:
establishing an algorithm in the personal computing and communications device for deriving treatment instructions from patient medical data; and
applying the algorithm to the first, second and third patient medical data to generate the patient treatment instructions.

36. The method of claim 25 wherein the generating step comprises extracting the patient treatment instructions from the third patient medical data.

37. The method of claim 25 wherein the expressing step comprises displaying the patient treatment instructions on a display screen associated with the personal computing and communications device.

38. The method of claim 25 wherein the expressing step comprises annunciating the treatment instructions with an audio output facility associated with the personal computing and communications device.

39. The method of claim 25 wherein:
the patient treatment instructions comprise a dosing command; and
the expressing step comprises communicating the dosing command to a dosing device associated with the patient.

40. The method of claim 39 wherein the dosing device is an implantable device.

41. The method of claim 25 wherein the third party medical data acquiring step and the furnish step are done in authenticated secure sessions using the communications capability of the personal computing and communications device.

42. The method of claim 41 further comprising initiating each one of the authenticated sessions with a biometric of the patient.

43. A mobile personal computing and communications device for distributed caregiving to a patient at a point-of-care, comprising:
means for storing first patient medical data in the personal computing and communications device at the point-of-care;
means for acquiring second patient medical data regarding a current condition of the patient with the personal computing and communications device at the point-of-care;
wireless communications means for acquiring third patient medical data from at least one remote caregiver in an authenticated session at the point-of-care, and for furnishing selected items from at least one of the first, second and third patient medical data to at least one remote caregiver in an authenticated session;
means for generating patient treatment instructions for the current condition with the personal computing and communications device from the third patient medical data; and
means for expressing the patient treatment instructions with the personal computing and communications device at the point-of care;
wherein the mobile personal computing and communications device is of a size, weight and form factor suitable for mobile use by the patient.

44. A mobile personal computing and communications device comprising:
a unit having a size, weight and form factor suitable for mobile use by the user;
a processor capability contained within the unit and having sufficient processing power for executing computer programs that are designed for personal computers;
a user interface functionally integrated with the processor capability;
a cellular communications capability contained within the unit;
a broadband wireless communications capability contained within the unit;
a wireless personal area network capability contained within the unit;
a security capability contained within the unit and functionally integrated with the processor capability, the cellular communications capability, and the broadband wireless capability for providing an authenticated login at the user interface and secure wireless communications with at least the broadband wireless communications capability;
a global positioning system capability functionally integrated with the processor capability;
an optical scanner capability functionally integrated with the processor capability;
a camera capability functionally integrated with the processor capability and the cellular communications capability;
a multiple-axes accelerometer functionally integrated with the processor capability and tightly mechanically coupled to the unit; and
a storage medium readable by the processor subsystem and comprising executable code.

45. The mobile personal computing and communications device of claim 44 wherein the storage medium comprises executable code for:
detecting via the accelerometer acceleration of the unit greater than a first threshold indicative of possible injury to a user of the personal computing and communications device;
detecting failure of the user to indicate no injury via the user interface in response to the first threshold acceleration detection step; and
requesting assistance in response to the failure detecting step, using any of the cellular communications capability, the broadband wireless communications capability, and the wireless personal area network capability.

46. The mobile personal computing and communications device of claim 45 wherein the executable code for requesting assistance further comprises executable code for:
furnishing a GPS location using the global positioning system capability; and
furnishing a visual image using the camera capability.

47. The mobile personal computing and communications device of claim 45 wherein the executable code for detecting failure of the user to indicate no injury comprises executable code for requiring at least one of an authenticated login at the user interface to indicate no injury and a user biometric via the optical scanner capability.

48. The mobile personal computing and communications device of claim 45 wherein the accelerometer is a three-axes accelerometer.

49. The mobile personal computing and communications device of claim 45 further comprising a hard drive, wherein the storage medium further comprises executable code for:
detecting via the accelerometer acceleration of the unit greater than a second threshold indicative of possible injury to the hard drive; and
securing the hard drive in response to the second threshold acceleration detecting step.

50. A method for proffering an authenticated medical directive of a person, comprising:
initiating a first authenticated session on a personal computing device with a biometric of the person, the personal computing device having a size, weight and form factor suitable for mobile use by the person;
storing a medical directive on the personal computing device during the patient-authenticated session;
during the patient-authenticated session, securing the stored medical directive for access by a proxy, the proxy being identified by a biometric of the proxy;
initiating a second authenticated session on the personal computing device with the biometric of the proxy; and
expressing the secured medical directive during the second authenticated session.

51. The method of claim 50 wherein the personal computing device has a wireless communications capability, further comprising:
preparing a confirmation version of the medical directive;
communicating the confirmation version of the medical directive to remote storage via the communications capability of the personal computing device; and
adjudicating credibility of the medical directive from the secured medical directive and from the confirmation version of the medical directive on the remote storage.
